(19) United States
(12) Patent Application Publication
(10) Pub. No.: US 2019/0142577 A1
(43) Pub. Date: May 16, 2019

(54) INTRAOCULAR LENS AND ASSOCIATED DESIGN AND MODELING METHODS

(71) Applicant: Theramedice LLC, Buffalo Grove, IL (US)

(72) Inventor: Jihong XIE, Aliso Viejo, CA (US)

(73) Assignee: Theramedice LLC, Buffalo Grove, IL (US)

(21) Appl. No.: 16/097,390

(22) PCT Filed: Apr. 26, 2017
(86) PCT No.: PCT/US2017/029600

§ 371 Date: Oct. 29, 2018

Related U.S. Application Data
(60) Provisional application No. 62/332,186, filed on May 5, 2016.
(60) Provisional application No. 62/332,675, filed on May 6, 2016.

(51) Int. Cl. A61F 2/16

(52) U.S. Cl. CPC A61F 2/1656; A61F 2240/001; A61F 2/1618

(57) ABSTRACT

A multifocal IOL (M-IOL) has a phase-altering characteristic that can control the diffraction and interference of light propagating there through to effect multifocality and extended depth of focus (EDOF). The embodied IOLs include engineered, discrete phase profiles on one or both of the anterior and posterior surfaces of the lens to intentionally manipulate the light in a designated manner. A design method for defining the discrete phase profile on the lens surface. The engineered phase profile is constructed by concentric annular zones having an abrupt step jump at the trailing circumferential edge of each zone. An optical modeling method to simulate the optical performance of the embodied IOLs in an optical ray tracing environment.

### RELATED APPLICATION DATA

The instant application claims priority to U.S. provisional application Ser. No. 62/332,186 filed May 5, 2016 and Ser. No. 62/332,675 filed May 6, 2016, the subject matters of which are incorporated by reference in their entireties.

### BACKGROUND

Aspects and embodiments of the invention pertain to intraocular lenses (IOLs) and methods for designing and modeling IOLs; more particularly to multifocal and/or extended depth of focus (EDOF) IOLs and associated methods; most particularly to such IOLs having discrete surface phase structures enabling multifocality and/or EDOF, and associated methods.

Multifocal IOLs exhibit multiple distinct diopter powers, which optically simultaneously focus images on the user's retina for objects at different distances. Extended depth of focus (EDOF) IOLs provide an extended range over which an object scene can be viewed in focus than that provided by a monofocal IOL. Such multifocality and EDOF aids users in regaining functional near and distance vision, and may alleviate presbyopia after cataract surgery.

The benefits and advantages provided by improved multifocal and EDOF IOLs can be realized by the embodied invention. Methods for designing and evaluating the embodied multifocal and EDOF IOLs are disclosed herein below. Several design examples generated from the embodied design and evaluation methods are further disclosed.

Multifocal lenses use either refractive optics or a combination of refractive/diffractive design to give the lens multiple (e.g., two, three, or more) foci. Conventional diffractive multifocal lenses utilize blazed diffractive gratings (such as a saw-like surface facet) to direct energy into several diffraction orders. The spatial frequency (i.e., inverse of grating period) of the diffractive grating determines the focus of each diffraction order, and the step height at the saw-like edge determines the energy distribution among different diffraction orders. For some conventional diffractive bifocal lenses the grating is generally designed with a single fixed spatial frequency, and the step height is generally designed to be less than a half wavelength, so that 80% of the incident light is split between distance and near focus, and the remaining 20% incident light is spread out to other diffraction orders that are not used for vision. For some conventional trifocal diffraction lenses, the grating is also designed with a single fixed frequency, but the step heights alternate between high and low between adjacent zones (e.g., step height above 0.5 wavelength and below 0.5 wavelength alternatively), and by doing that, the design achieves an approximately 85% energy split between distance, intermediate, and near focus while the rest of the 15% incident light goes to the diffraction orders that are not used for vision.

None of the existing design methods for diffractive multifocal IOLs are able to provide the full freedom to manipulate the phase distribution on the diffractive surface to the energy distribution among usable diffraction orders and minimize the energy that goes to unusable diffraction orders. In the embodied invention, the concept of weighted local diffraction efficiency is introduced to maximize usage of the incident light in functional diffraction orders for vision and effectively distribute this energy among these orders to achieve multifocality and extended depth of focus.

### SUMMARY

An aspect of the embodied invention is a multifocal IOL (M-IOL). In an embodiment, a lens having a phase-altering characteristic can control the diffraction and interference of light propagating therethrough to effect multifocality and extended depth of focus (EDOF). The embodied IOLs include engineered, discrete phase profiles on one or both of the anterior and posterior surfaces of the lens to intentionally manipulate the light in a designated manner.

In a non-limiting embodiment, the discrete phase profiles are provided by structural step profiles each having a maximum step height, h, on a scale of 0 to ˜2 wavelengths, λ (where λ is the primary IOL design wavelength). Each of the step profiles are incorporated in a respective plurality, m, of contiguous, annular optical zones each defined by a radius, rₘ, on a surface of the lens and extending from the lens center out towards the periphery. As such, each optical zone, m, will exhibit multiple (n) diffraction orders manifested as ‘Add-Powers.’ The total effective optical area of the lens is defined as the combined area of the m optical zones.

An exemplary multifocal intraocular lens (M-IOL) includes a lens body having an anterior surface and a posterior surface, wherein at least one of the anterior and the posterior surface is characterized by a discrete phase profile comprising a plurality, m (m=0, 1, 2, 3, . . . ), of contiguous annular, diffractive optical zones each characterized by a radius, rₘ, and a step height, hₘ, at each respective rₘ, wherein at least some values of hₘ may not be equal to hₘ₊ₓ (x=1, 2, 3, . . . ), wherein rₘ=(2mλf)^{1/2}, where λ is the design wavelength and f is the focal length (1000 mm/Add-Power) corresponding to a selected Add-Power for the IOL, further wherein [symbol] ₙ is the diffraction efficiency in a particular optical zone, m, for the n^{th} diffraction order (n=0, 1, 2, 3 . . . ) corresponding to the n^{th} Add-Power in that particular optical zone, m, wherein

[symbol]

_{n,m}=[sin(π(k−n))/(π(k−n))]²=SINC²(π(k−n))+f(rₘ) (2)

where: f(rₘ) for k=(n₂−n₁)hₘ/λ is a factor for adjusting the step height, hₘ, where (n₂−n₁) is the refractive index difference between a non-lens medium and the lens optical zone (diffractive) medium, wherein the step height, hₘ, can be determined from the designated [symbol] _{n, m}, further wherein an overall energy distribution over a total effective (diffractive) optical area of the IOL is represented as a weighted summation of a local diffraction efficiency [symbol] _{n, m} of the particular optical zone, m (in which n is the diffraction order corresponding to the Add Powerₙ) in that m^{th} optical zone, wherein a weighting factor is determined by a surface area ratio, Rₘ, between the individual optical zone, m, and the total effective (diffractive) optical area of the IOL, where [symbol] ₙ=ΣRₘ [symbol] _{n,m} (m=1, 2, 3 . . . , n=0, 1, 2, 3 . . . ) and Rₘ=(area of the m^{th} annular optical zone)/(total effective (diffractive) optical area of IOL). In various non-limiting embodiments, the M-IOL may be further characterized by one or more of the following features, limitations, characteristics, and/or components, separately or in various combinations as a person skilled in the art would understand:

characterized in that [symbol] _{n,m} has a constant value for all of the optical zones, m, and Rₘ has a constant value for all of the optical zones, m;

characterized in that [symbol] _{n,m} has a variable value for all of the optical zones, m, and Rₘ has a constant value for all of the optical zones, m;

characterized in that [symbol] _{n,m} has a constant value for all of the optical zones, m and Rₘ has a variable value for all of the optical zones, m;

characterized in that [symbol] _{n,m} has a variable value for all of the optical zones, m, and Rₘ has a variable value for all of the optical zones, m;

characterized in that [symbol] _{n,m} has a variable value for all of the optical zones, m, and Rₘ has a variable value for all of the optical zones, m.

In determining [symbol] _{n,m} using Equation (2), f(rₘ) is an adjusting function for optimizing light distribution among usable diffraction orders and minimizing light spread in unusable diffraction order, and it provides the flexibility of not limiting the exact surface profile of m-th diffraction to spherical but being extended to aspheric or freeform. The function f(rₘ) is determined by the Fourier Transform of the exact phase profile for the m-th diffraction zone.

An aspect of the invention is a design method for defining the discrete phase profile on the lens surface. According to a non-limiting embodiment, the engineered phase profile is constructed by concentric annular zones having an abrupt step jump at the trailing circumferential edge of each zone. To minimize the spread of incident light into unusable diffraction orders as well as to flexibly distribute energy among usable diffraction orders so that effective multifocality and extended depth of focus can be achieved, the optimization of surface profile of concentric annular zones is not limited to a spheric surface, but can also be extended to conic, general aspheric, or freeform surface profiles. In addition the abrupt step jump at the trailing circumferential edge of each zone is not limited to a vertical profile, but can also be a sloped or curved surface profile.

An aspect of the invention is an optical modeling method to simulate the optical performance of the embodied IOLs in an optical ray tracing environment. In an exemplary embodiment, the method involves the establishment of an optical raytracing model eye that can simulate the optical performance of the eye with the IOL plugged in the model. The method further involves the construction of a user-defined surface that can be used to input the discrete surface phase profile in the optical raytracing model. The discrete phase surface profile is associated with user-defined functions that can adjust the phase parameter of each ray traced through the surface based on the designed local diffractive structure profile. The method more particularly involves the following steps: 1) trace rays with phase parameters modified by the diffractive surface to the exit pupil of the raytracing model and construct the true pupil function; 2) obtain the Optical Transfer Function (OTF); 3) obtain the modulation transfer function (MTF); 4) obtain the MTF at different defocus locations; 5) obtain the system Point Spread Function (PSF); 6) conduct imaging simulation.

### BRIEF DESCRIPTION OF THE DRAWINGS

FIG. 1: Schematic of discrete diffractive annular optical zones on lens surface.

FIG. 2: Cross sectional schematic of embodied diffractive lens illustrating 0^{th}, 1^{st}, and 2^{nd} diffraction orders corresponding to f₀, f₁, and f₂, further corresponding to baseline, add power 1, add power 2.

FIG. 3A: Type A energy distribution for 3.0 D add power bifocal design, between distance and near focus as a function of the change of pupil size; FIG. 3B: Type B energy distribution for 3.0 D add power bifocal design, between distance and near focus as a function of the change of pupil size.

FIG. 4A: Type A surface phase structure of the Bifocal IOL with 3.0 D add power (baseline refractive power subtracted); FIG. 4B: Type B surface phase structure of the Bifocal IOL with 3.0 D add power (baseline refractive power subtracted).

FIGS. 5A-H: Modulation Transfer Functions (MTFs) for 3.0 D add bifocal design for different pupil sizes and for Type A and Type B energy distributions.

FIGS. 6A-D: Image simulations for 3.0 D add bifocal design for different pupil sizes and for Type A and Type B energy distributions.

FIGS. 7A-D: Simulation of the through-focus performance of the 3.0 D add bifocal IOL for different pupil sizes and for Type A and Type B energy distributions.

FIGS. 8A-B: Surface phase structure of the trifocal IOL with 1.5 D and 3.0 D add power (baseline refractive power subtracted) for different pupil sizes and for Type A and Type B energy distributions.

FIGS. 9A-B: Energy distributions for 1.75 D and 3.5 D add trifocal design, between distance, intermediate, and near focus for different pupil sizes and for Type A and Type B energy distributions.

FIGS. 10A-F: Modulation Transfer Functions (MTFs) for 1.75 D and 3.5 D add trifocal Type A design.

FIGS. 11A-B: Imaging simulations for the trifocal design with 1.75 D and 3.5 D add trifocal Type A design.

FIG. 12: Simulation of the through-focus performance of the trifocal IOL Type A design.

FIG. 13: Surface phase structure of EDOF IOL with depth of focus extended beyond 2.5 D (baseline refractive power subtracted).

FIGS. 14A-B: Imaging simulations for the EDOF design and traditional monofocal IOL design.

FIG. 15: Simulation of the through-focus MTF performance of the EDOF IOL and the traditional monofocal IOL.

FIG. 16: Surface discrete phase structure of the alternate trifocal IOL (baseline refractive power subtracted).

FIG. 17: Simulation of the through-focus performance of the alternative trifocal IOL.

### DETAILED DESCRIPTION OF NON-LIMITING, EXEMPLARY EMBODIMENTS

Design methodologies for the embodied IOLs derive from the wave nature of light. Per Huygens's diffraction principle, light, as a wave, is described by wavelength, phase, and amplitude, and it presents phenomena of diffraction and interference as it propagates in/through/between a medium or media.

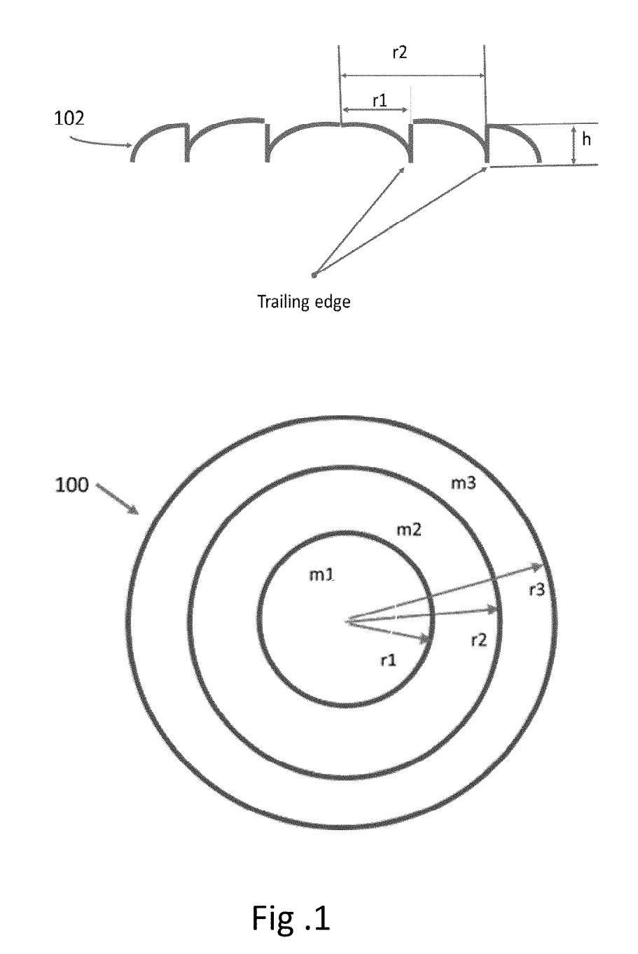

As illustrated in FIG. 1, a lens 100 having a phase-altering characteristic can control the diffraction and interference of light propagating there through to effect multifocality and EDOF. The discrete phase profiles are provided by structural step profiles 102 (top) each having a maximum step height, h, on a scale of 0 to ˜2 wavelengths, λ (where λ is the primary IOL design wavelength. Each of the step profiles 102 is incorporated in a respective plurality, m, of contiguous, annular optical zones each defined by a radius, rₘ, on a surface of the lens and extending from the lens center out towards the periphery, as illustrated in FIG. 1 (bottom). The total effective optical area of the lens is defined as the combined area of the m optical zones.

The concentric annular zones, m, are characterized by two major parameters, e.g., the location/radius, rₘ, of the ring and the height, h, of the abrupt step jump (peak height phase departure). These parameters are determined as follows:

The radius, rₘ, of the m^{th} ring is given by

rₘ=(2mλf)^{1/2} (1)

where m=0, 1, 2 . . . (integer values), λ is the primary IOL design wavelength, and f is the focal length corresponding to the ‘add power’ of the intended multifocality; i.e., f=1000 mm/add power).
The step height, h, at the trailing circumferential edge of each annular optical zone is given by

[symbol]

ₙ=[sin(π(k−n))/(π(k−n))]²=SINC²(π(k−n))+f(rₘ), (2)

in which [symbol] ₙ is diffraction efficiency for the n^{th} diffraction order (n=0, 1, 2, 3 . . . ), k=(n₂−n₁)h/λ is a factor for adjusting phase jump, (n₂−n₁) is the refractive index difference between the medium in which the lens is occupied (e.g., user's eye, or air if not implanted) and the optical zone (grating) material, and h is the step height, which can be solved from the designated [symbol] ₙ

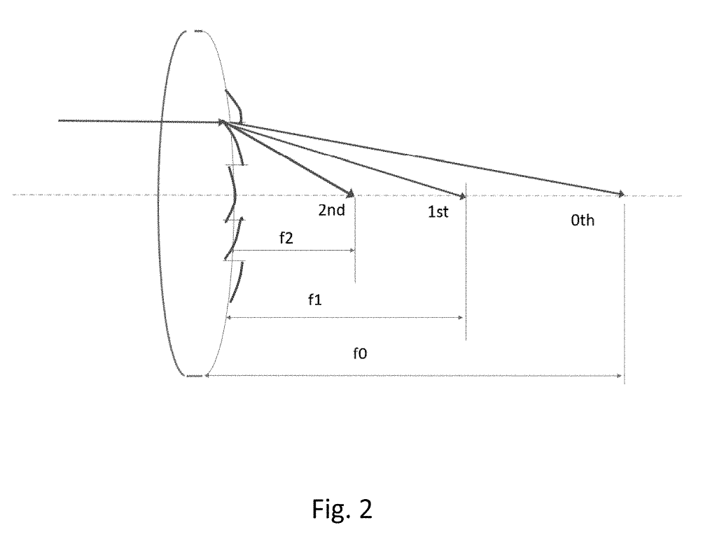

Equation (2) describes how diffraction efficiencies, [symbol] ₙ, are associated with each diffraction order (n) of interest in each annular optical zone, m. Diffraction efficiency is a parameter that quantitatively describes how light energy is distributed between different foci (add-powers) in each optical zone. This is schematically illustrated in FIG. 2. In FIG. 2, 0^{th}, 1^{st}, and 2^{nd} diffraction orders correspond to the lens' base power (f₀), first add-power (f₁), and second add-power (f₂) in each optical zone, m.

In Equation (2), f(rₘ) is an adjusting function for optimizing light distribution among usable diffraction orders and minimizing light spreading into unusable diffraction orders, and it provides flexibility for the surface profile of the m-th diffraction zone not to be limited to spherical but can be extended to aspheric or freeform. f(rₘ) is related to the Fourier Transform of the exact phase profile for the m-th diffraction zone.

Equation (2) is derived from Fraunhofer diffraction calculations on generalized gratings, and in the embodied invention each optical zone, m, is treated as a particular single local grating.

The derivation of equations (1) and (2) are set forth in Appendix 1 at the end of the specification.

For the total optical area of the lens surface, the overall energy distribution among different diffraction orders, n (each individual diffraction order, n, corresponding to each individual focus or add-power in each optical zone, m), is treated as the weighted summation of individual diffraction efficiencies in each local zone (denoted as [symbol] _{n,m}, in which n represents the diffraction order and m represents the m^{th} annular optical zone. The weighting factor is determined by the surface area ratio, Rₘ, between the individual optical zone and the total effective optical area as follows:

[symbol]

ₙ=ΣRₘ [symbol] _{n,m},

where [symbol] _{n,m} is the local diffraction efficiency of the m^{th} zone and
Rₘ=(area of the m^{th} annular optical zone)/(total effective (diffractive) optical area of IOL).

To achieve a desired energy distribution among different foci, or to achieve extended depth of the focus, the surface phase profile is optimized via an appropriate combination of local optical zone diffraction efficiency [symbol] _{n,m} and the weighting factor Rₘ. According to exemplary embodiments, four approaches, summarized in Table 1 below, are used to design the embodied diffractive, multifocal and/or EDOF lenses, examples of which are described herein below.

TABLE 1

| | Local diffraction | Surface area |
|---|---|---|
| Approach | efficiency ( [symbol] ) | ratio (Rₘ) |
| I | fixed for all zones | fixed for all zones |
| II | varied among zones | fixed for all zones |
| III | fixed for all zones | varied among zones |
| IV | varied among zones | varied among zones |

According to an illustrative embodiment, an optical modeling method is used to simulate the optical performance of the embodied IOLs in an optical ray tracing environment. The method involves the establishment of an optical raytracing model eye that can simulate the optical performance of the eye with the IOL plugged in the model.

The method further involves the construction of a user-defined surface that can be used to input the discrete surface phase profile in the optical raytracing model. The discrete phase surface profile is associated with user-defined functions that can adjust the phase parameter of each ray traced through the surface based on the designed local diffractive structure profile.

The method further relies on incoherent imaging frequency response analysis techniques to simulate the optical performance of the design. The fundamental theory is summarized in Appendix 2 at the end of the specification. The metrics for evaluating the optical image quality include the Point Spread Function (PSF), the Modulation Transfer Function (MTF), and Imaging Simulation. The method more particularly involves the following steps: 1) trace rays with phase parameters modified by the diffractive surface to the exit pupil of the raytracing model, and construct the true pupil function; 2) obtain the Optical Transfer Function (OTF), which is calculated as the auto-correlation of the pupil function that is based on the raytracing data at the exit pupil; 3) obtain the modulation transfer function (MTF), which is the modulation of the OTF, which describes the image contrast degradation at various spatial frequencies from object to image; 4) obtain the MTF at different defocus locations, which describes the through-focus performance of the design; 5) obtain the system Point Spread Function (PSF) via inverse Fourier transforms of the OTF; 6) conduct imaging simulation by taking the convolution of the PSF and the object (inverse Fourier transform of the product of the OTF and the spectrum of the object).

Non-limiting embodiments include four exemplary IOL designs based on approaches I-IV in Table I above, as follow.

### Example 1 (Approach I)

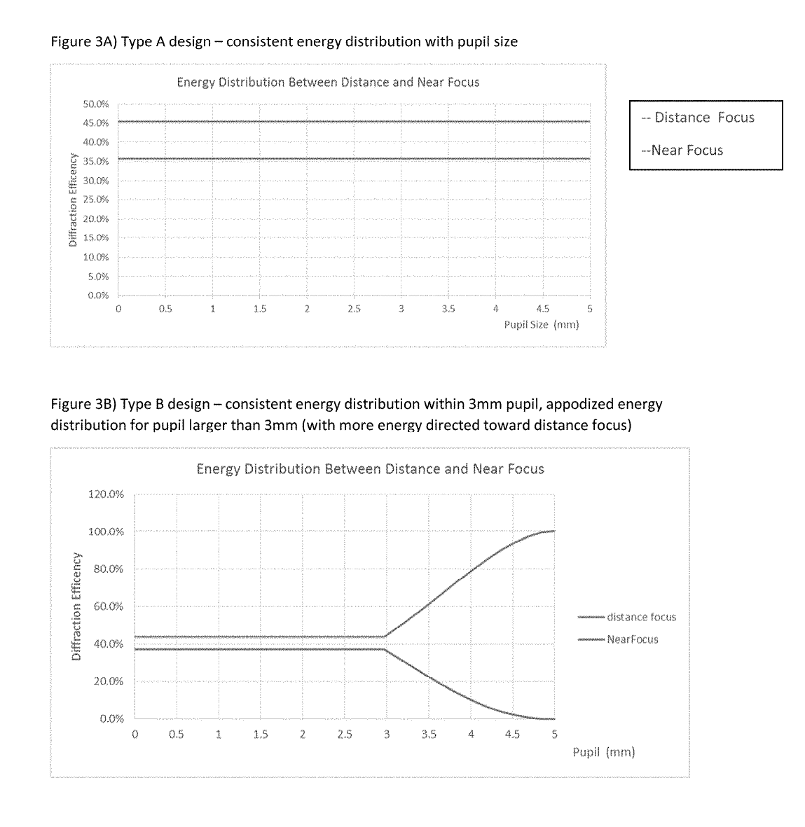

A bifocal IOL with 3.0 diopter (D) add power, which corresponds to the distance and near focus, respectively. This design form has a diffractive structure with consistent surface area ratio, Rₘ, for each diffraction zone, and uniformly (monotonically) decreasing diffraction efficiency (i.e., uniformly (monotonically) decreasing step height). Table 2 and FIGS. 3A-7D disclose and illustrate the design parameters and performance predictions.

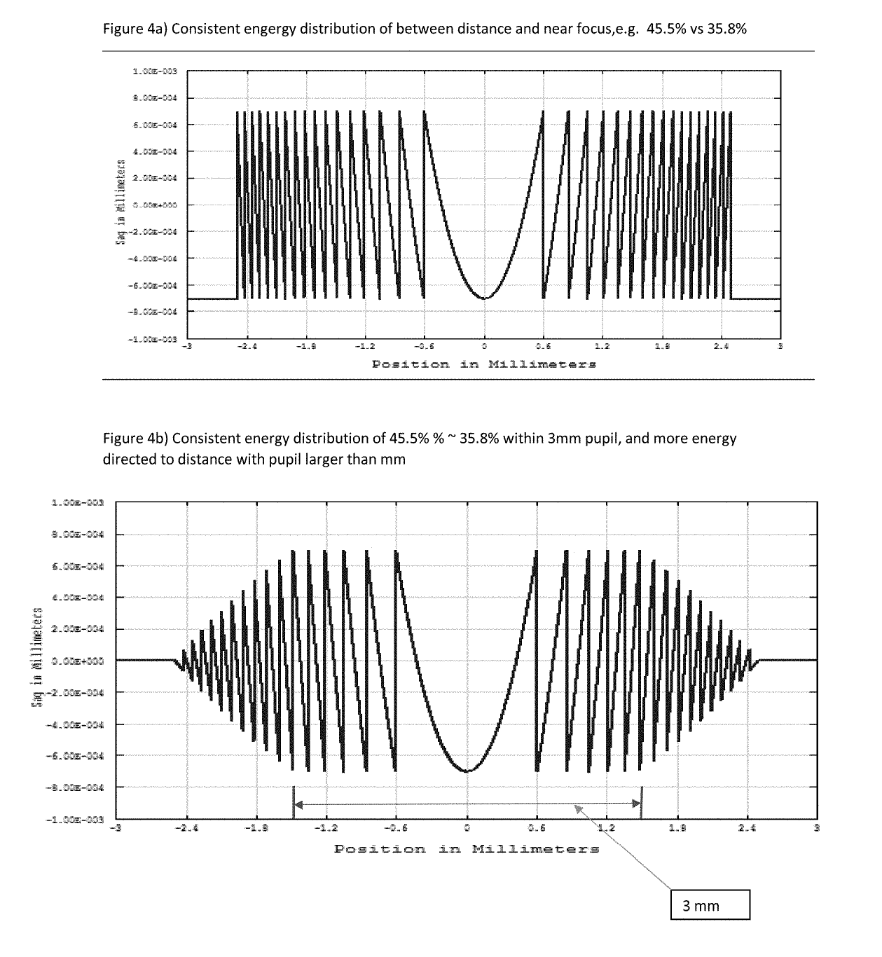

The bifocal IOL is designed with 3.0 D add-power. This design form includes Type A and Type B designs. Type A design has a consistent 45.5%/35.8% energy distribution between distance and near focus at all pupil sizes, as illustrated in FIG. 3A. Type B has consistent distance/near energy distribution of 45.5%/35.8% for the center 3 mm region, and a gradual, uniformly changing energy distribution with pupil size larger than 3 mm, with more energy directed towards the distance focus with increase in pupil size, as illustrated in FIG. 3B. FIGS. 4A, 4B show the discrete surface phase structures, and Table 2 lists the specified design parameters for the ring locations and step heights at the rings' trailing edges. The values specified in Table 2 particularly apply to lens materials having a refractive index of 1.52 at 550 nanometer wavelength. For materials with other refractive indices, the step height will need to be adjusted as follows:

h′=h*C

C=0.184/(n′−1.336),

in which
h′ is the adjusted step height for different refractive index n′,
h is the step height specified in Table 2,
C is an adjusting coefficient,
n is the material refractive index corresponding to Table 2,
n′ is the different material refractive index. The embodied design is enabled for materials of refractive index from 1.40-1.58.

TABLE 2
Discrete surface structure for diffractive bifocal IOL
Type A design

| | rⱼ | Step Height, hⱼ |
|---|---|---|
| Zone #, j | (mm) | (um) |
| 0 | 0 | 1.4049 |
| 1 | 0.6055 | 1.4049 |
| 2 | 0.8563 | 1.4049 |
| 3 | 1.0488 | 1.4049 |
| 4 | 1.2111 | 1.4049 |
| 5 | 1.3540 | 1.4049 |
| 6 | 1.4832 | 1.4049 |
| 7 | 1.6021 | 1.4049 |
| 8 | 1.7127 | 1.4049 |
| 9 | 1.8166 | 1.4049 |
| 10 | 1.9149 | 1.4049 |
| 11 | 2.0083 | 1.4049 |
| 12 | 2.0976 | 1.4049 |
| 13 | 2.1833 | 1.4049 |
| 14 | 2.2657 | 1.4049 |
| 15 | 2.3452 | 1.4049 |
| 16 | 2.4221 | 1.4049 |
| 17 | 2.4967 | 1.4049 |

Type B design

| | rj | Step Height, hj |
|---|---|---|
| Zone #, j | (mm) | (um) |
| 0 | 0.0000 | 1.4049 |
| 1 | 0.6055 | 1.4049 |
| 2 | 0.8563 | 1.4049 |
| 3 | 1.0488 | 1.4049 |
| 4 | 1.2111 | 1.4049 |
| 5 | 1.3540 | 1.4049 |
| 6 | 1.4832 | 1.4049 |
| 7 | 1.6021 | 1.2772 |
| 8 | 1.7127 | 1.1495 |
| 9 | 1.8166 | 1.0217 |
| 10 | 1.9149 | 0.8940 |
| 11 | 2.0083 | 0.7663 |
| 12 | 2.0976 | 0.6386 |
| 13 | 2.1833 | 0.5109 |
| 14 | 2.2657 | 0.3832 |
| 15 | 2.3452 | 0.2554 |
| 16 | 2.4221 | 0.1277 |
| 17 | 2.4967 | 0.0000 |

The performance of Example 1 IOL was evaluated by the embodied modeling and analysis techniques disclosed herein above.

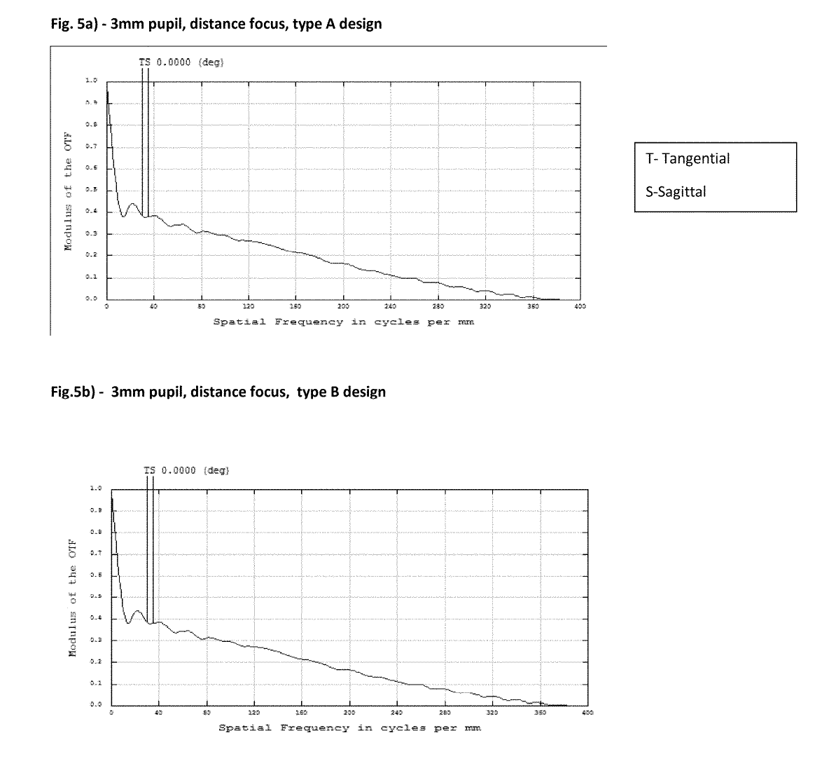

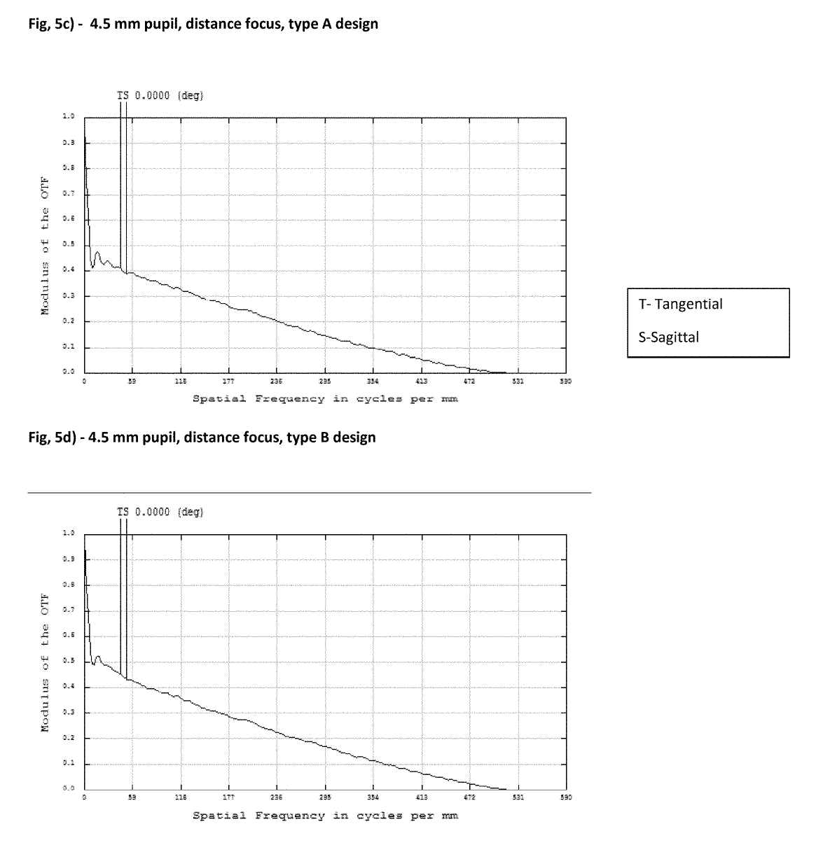

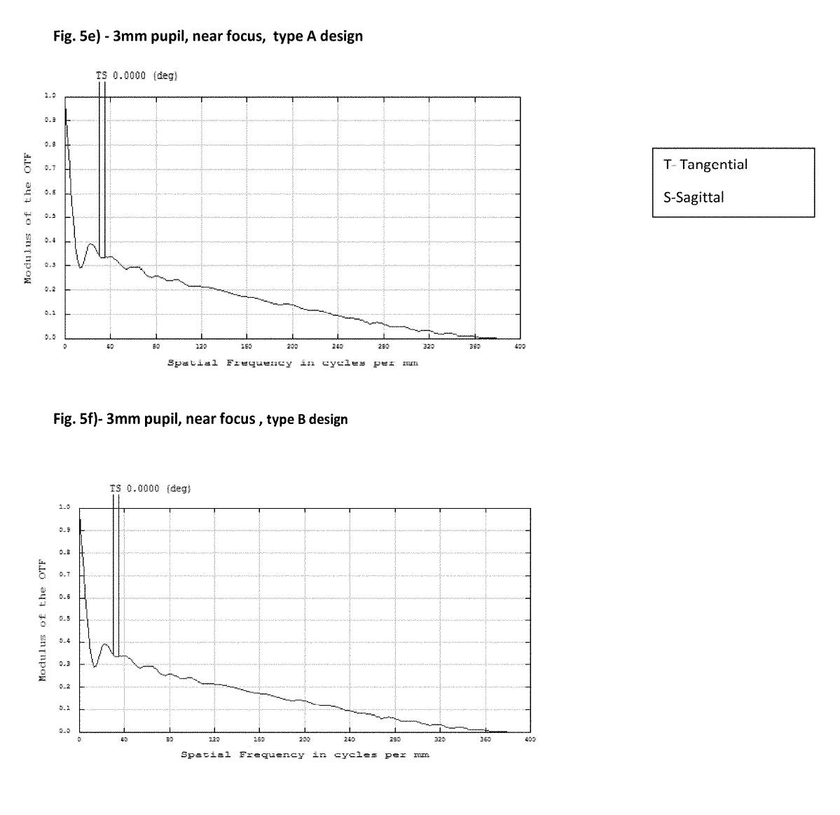

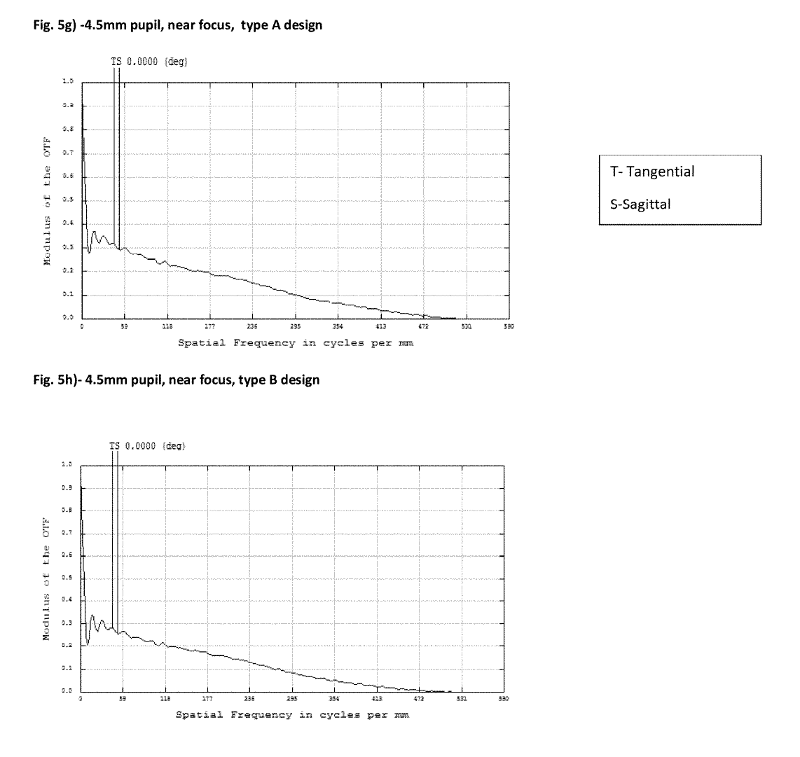

FIGS. 5A-5H show the Modulation Transfer Functions (MTFs) with a 3 mm pupil (Type A (FIGS. 5A, 5E) and Type B (FIGS. 5B, 5F)) and a 4.5 mm pupil (Type A (FIGS. 5C, 5G) and Type B (FIGS. 5D, 5H) at distance and near foci (tangential and sagittal planes are shown).

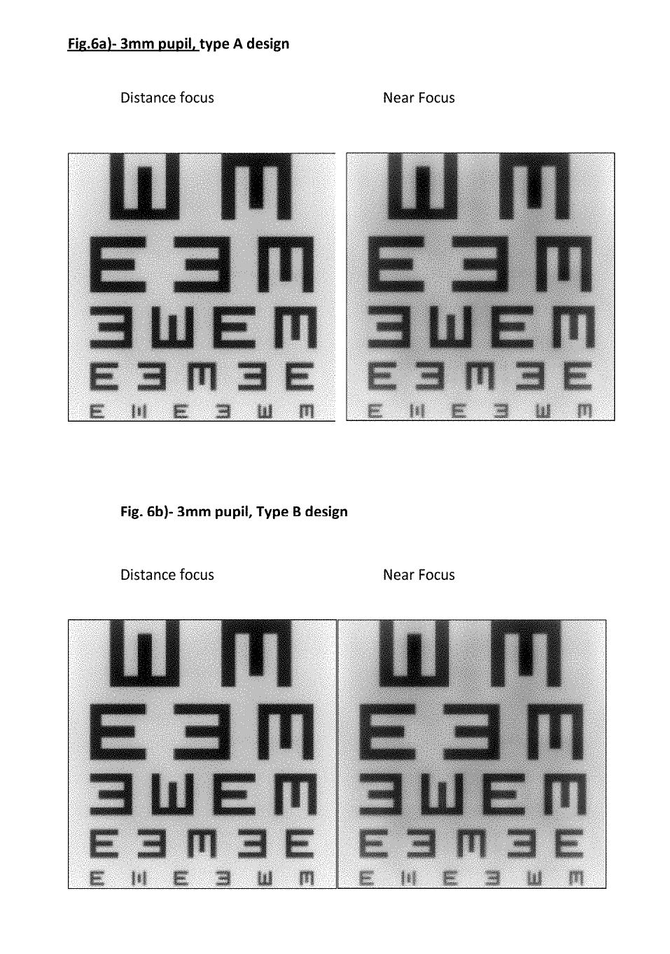

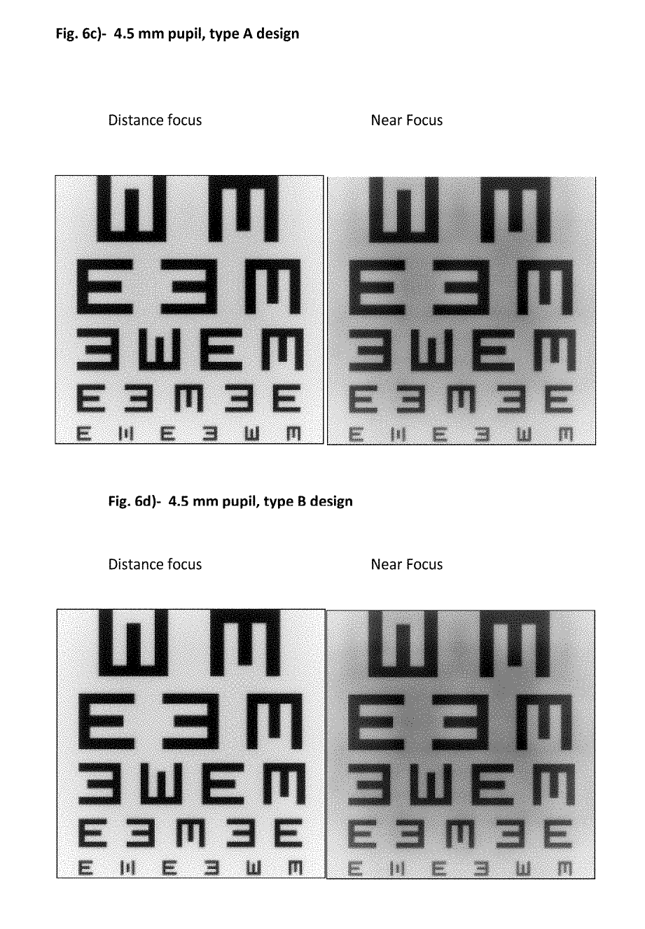

FIGS. 6A-D show the simulation of imaging at the model eye retina for distance focus and near focus with two pupil sizes (3 mm, 4.5 mm).

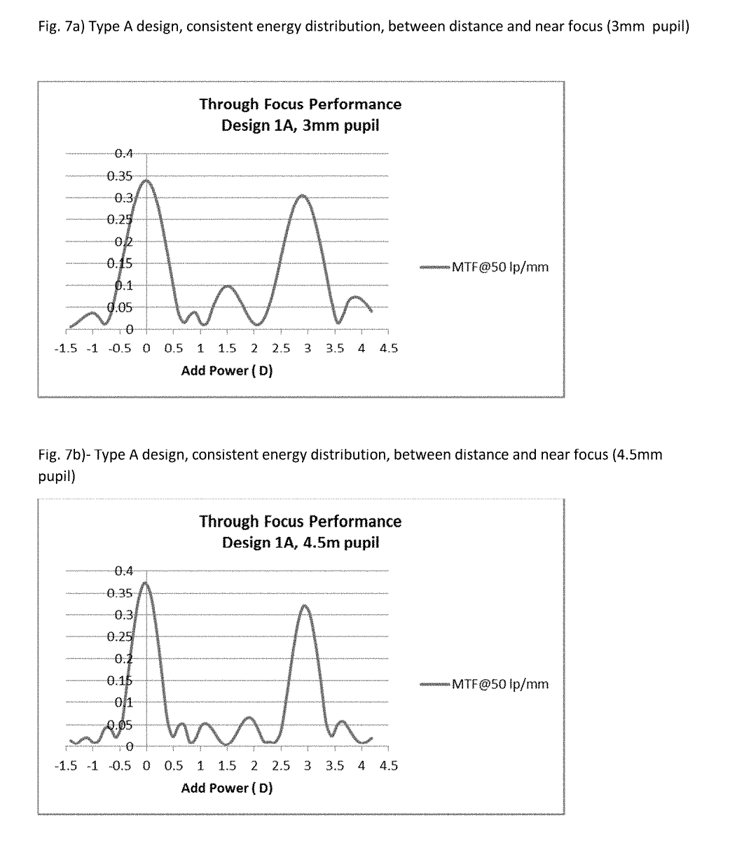

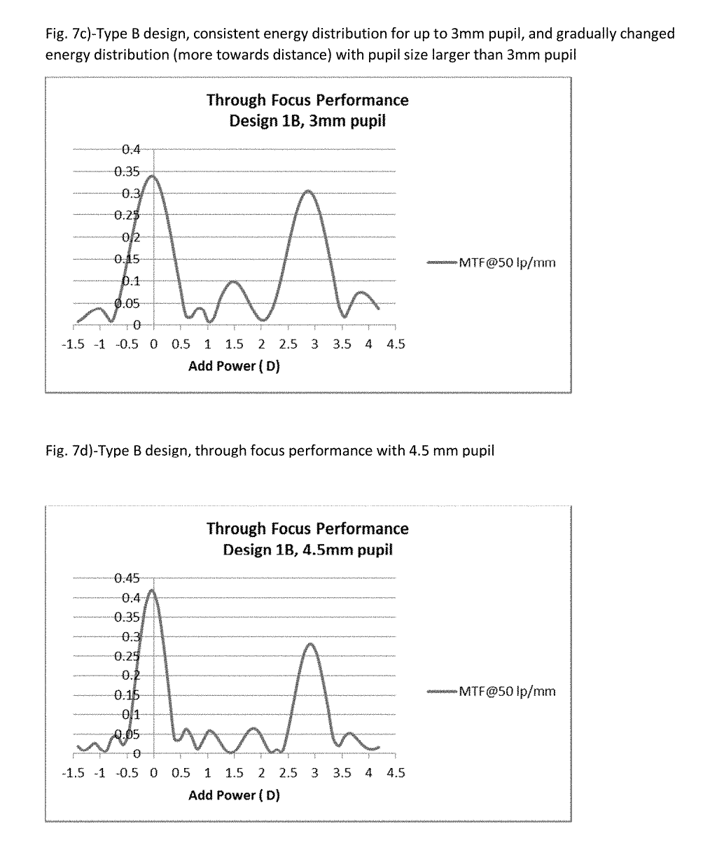

FIGS. 7A-D show the simulated through-focus MTF curves with two pupil sizes (3 mm, 4.5 mm). Type A and Type B are designed with identical optical performance for a 3 mm pupil; however, while the pupil becomes larger than 3 mm, the Type A (FIGS. 7A, 7B) design maintains consistent performance and the Type B (FIGS. 7C, 7D) design directs more light energy towards the distance focus; e.g., the MTF curve becomes higher at distance focus and lower at near focus.

### Example 2 (Approach II)

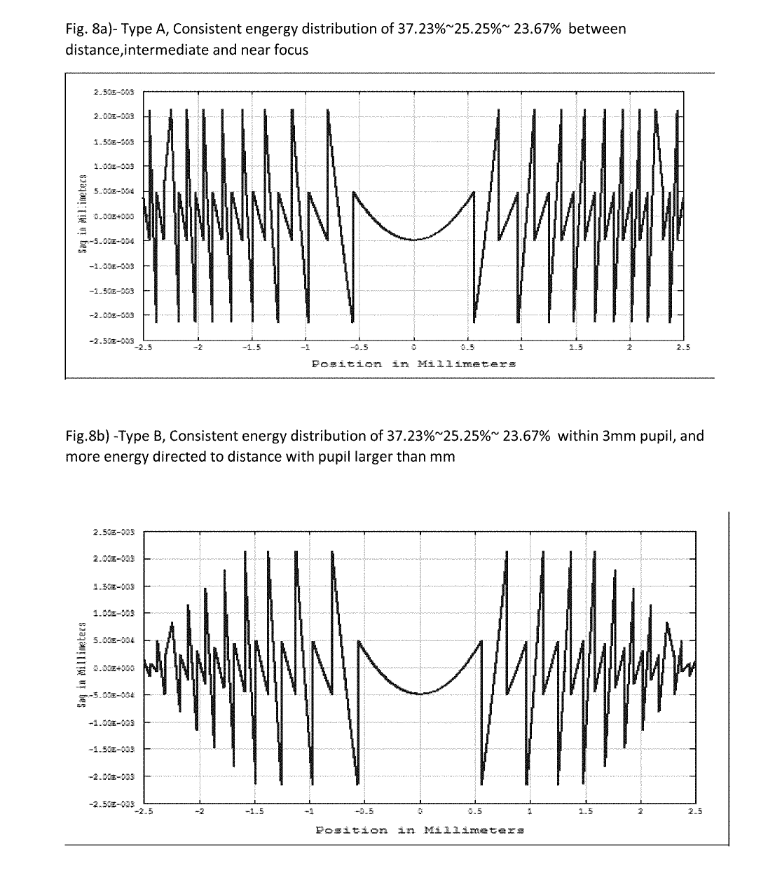

A trifocal IOL with 1.75 D and 3.5 D add powers, which correspond to the distance, intermediate, and near focus, respectively. This design form has a diffractive structure with consistent surface area ratio for each diffraction zone, but varying diffraction efficiencies for adjacent zones (alternate high and low step heights). Table 3 and FIGS. 8A-12 disclose and illustrate the design parameters and performance predictions.

The trifocal IOL is designed with two distinct add powers; e.g., 1.75 D and 3.50 D, to provide distance, intermediate, and near vision. Similar to Example 1, this design form can include Type A and Type B designs. Type A design has consistent 37.2%, 25.3%, and 23.7% energy distributions between distance, intermediate, and near focus at all pupil sizes. Type B has consistent distance/intermediate/near energy distributions of 37.2%/25.3%/23.7% for only the center 3 mm region, but gradually changing energy distribution with pupil size increasing from 3 mm to 5 mm, with more towards the distance and intermediate foci, while the pupil size increases.

FIGS. 8A, 8B illustrate the discrete surface phase structures for Type A and Type B, respectively, and Table 3 the specified design parameters for the ring locations and step heights at the ring's trailing edges. The parameters specified in Table 3 particularly applied to materials having a refractive index of 1.52 at 550 nanometer wavelength. For materials with other refractive indices, the step height will need to be adjusted as follows:

h′=h*C,

C=0.184/(n′−1.336),

in which
h′ is the adjusted step height for different refractive index n′,
h is the step height specified in Table 3,
C is an adjusting coefficient,
n is the material refractive index corresponding to Table 3,
n′ is the different material refractive index. The embodied design is enabled for materials of refractive index from 1.40-1.58.

TABLE 3
Discrete surface structure

| Trifocal IOL, Type A design | | | Trifocal IOL, Type B design | | |
|---|---|---|---|---|---|
| | | Step | | | Step |
| | rj | Height, hj | | rj | Height, hj |
| Zone #, j | (mm) | (um) | Zone #, j | (mm) | (um) |
| 0 | 0.0000 | 0.9715 | 0 | 0.0000 | 0.9715 |
| 1 | 0.5606 | 4.3043 | 1 | 0.5606 | 4.3043 |
| 2 | 0.7928 | 0.9715 | 2 | 0.7928 | 0.9715 |
| 3 | 0.9710 | 4.3043 | 3 | 0.9710 | 4.3043 |
| 4 | 1.1212 | 0.9715 | 4 | 1.1212 | 0.9715 |
| 5 | 1.2536 | 4.3043 | 5 | 1.2536 | 4.3043 |
| 6 | 1.3732 | 0.9715 | 6 | 1.3732 | 0.9715 |
| 7 | 1.4832 | 4.3043 | 7 | 1.4832 | 4.3043 |
| 8 | 1.5856 | 0.9715 | 8 | 1.5856 | 0.8967 |
| 9 | 1.6818 | 4.3043 | 9 | 1.6818 | 3.6421 |
| 10 | 1.7728 | 0.9715 | 10 | 1.7728 | 0.7473 |
| 11 | 1.8593 | 4.3043 | 11 | 1.8593 | 2.9799 |
| 12 | 1.9420 | 0.9715 | 12 | 1.9420 | 0.5978 |
| 13 | 2.0213 | 4.3043 | 13 | 2.0213 | 2.3177 |
| 14 | 2.0976 | 0.9715 | 14 | 2.0976 | 0.4484 |
| 15 | 2.1712 | 4.3043 | 15 | 2.1712 | 1.6555 |
| 16 | 2.2424 | 0.9715 | 16 | 2.2424 | 0.2989 |
| 17 | 2.3115 | 4.3043 | 17 | 2.3115 | 0.9933 |
| 18 | 2.3785 | 0.9715 | 18 | 2.3785 | 0.1495 |
| 19 | 2.4437 | 4.3043 | 19 | 2.4437 | 0.3311 |
| 20 | 2.5071 | 0.9715 | 20 | 2.5071 | 0.0000 |

The performance of Example 2 IOL was evaluated by the embodied modeling and analysis techniques disclosed herein above.

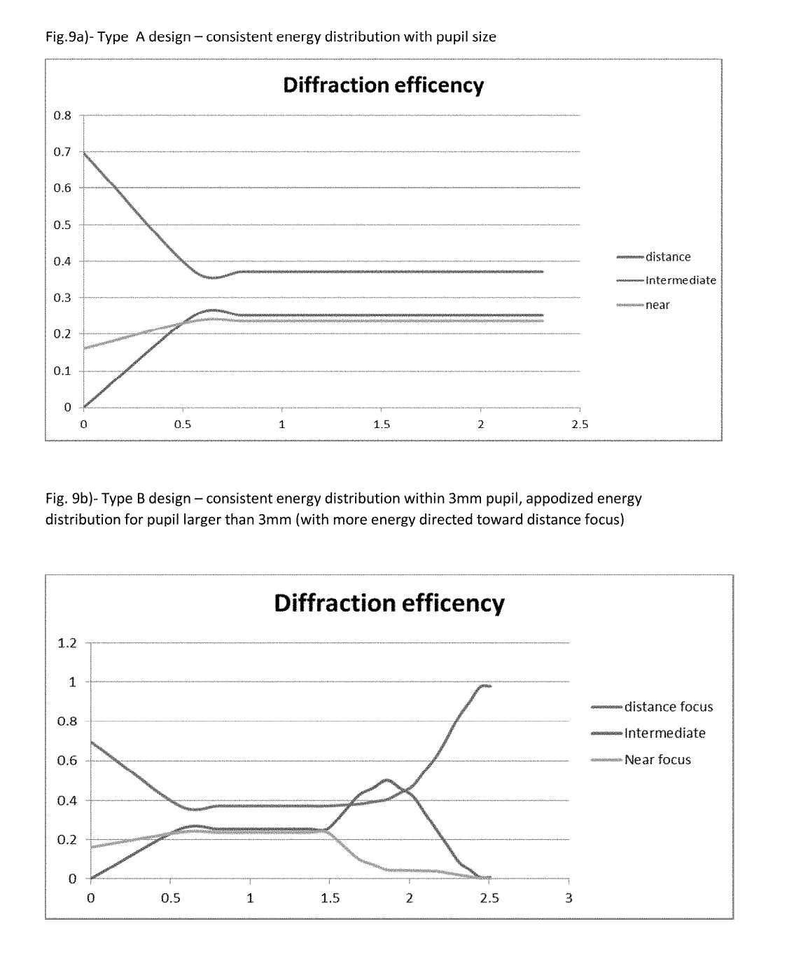

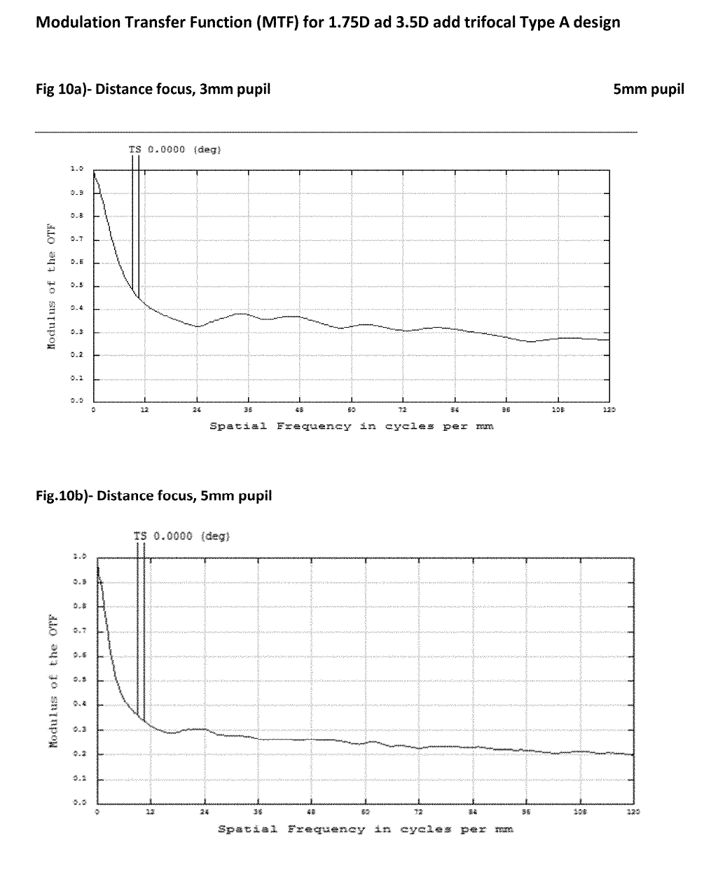

FIGS. 9A, 9B show the energy distribution of distance/intermediate/near foci for both Type A and Type B designs, respectively. Then for Type A design, FIGS. 10A-10F show the Modulation Transfer Functions (MTFs) at distance, intermediate, and near focus (tangential and sagittal planes are shown).

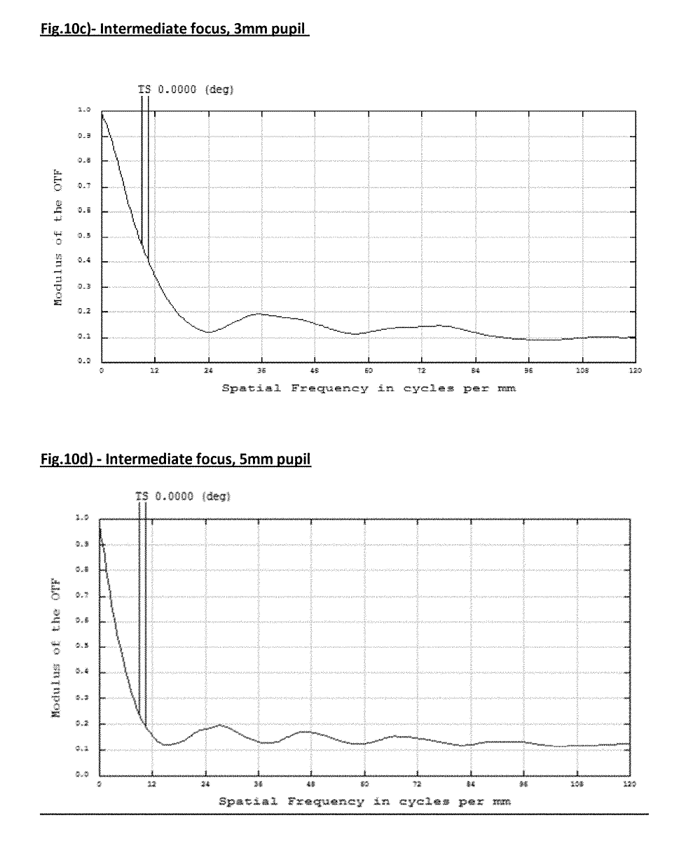

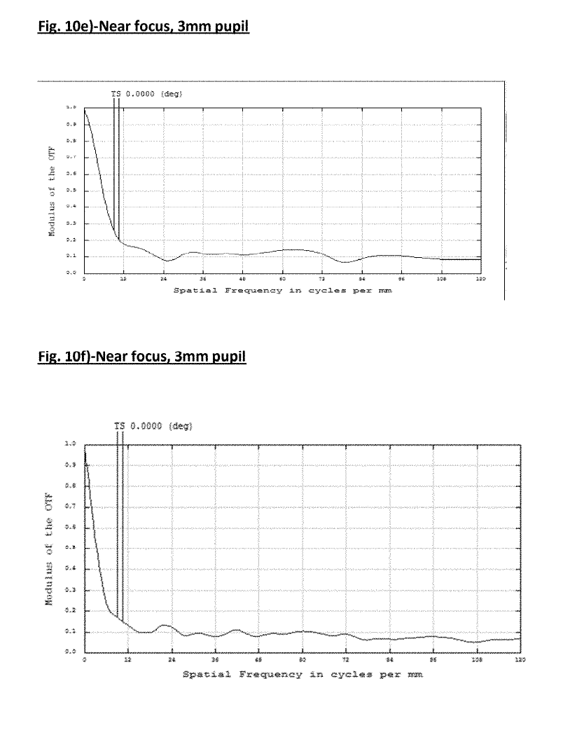

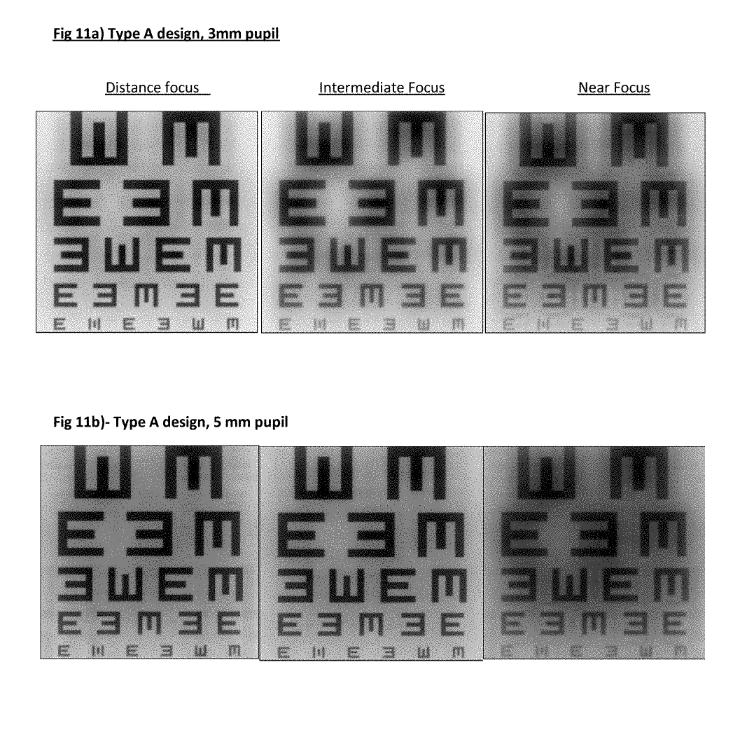

FIGS. 11A, 11B show the simulation of imaging at the model eye retina for distance, intermediate, and near focus with two pupil sizes (3 mm, 5 mm), respectively.

FIG. 12 shows the simulated through-focus MTF curves with two pupil sizes.

### Example 3 (Approach III)

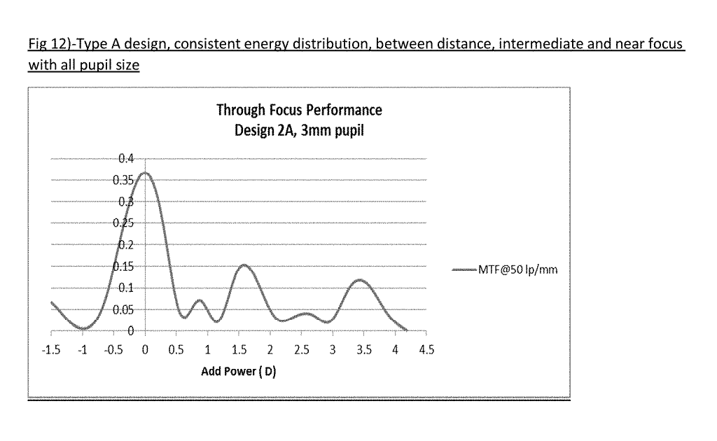

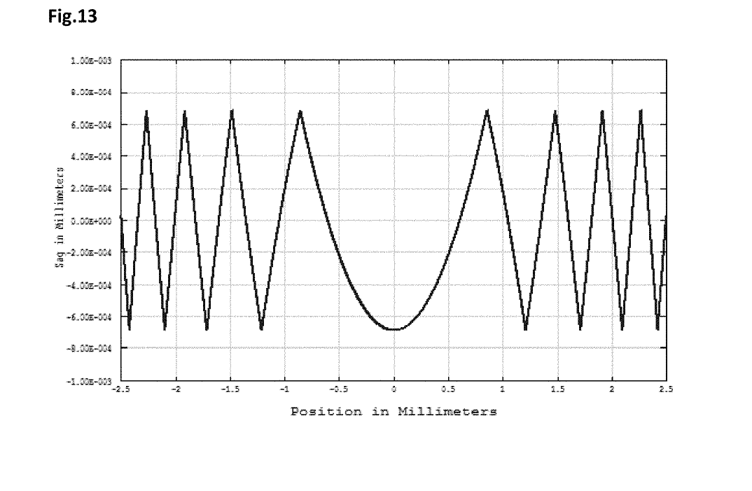

An extended Depth of Focus IOL (EDOF IOL) with the continuous depth of focus extended to larger than 2.5 D (compared to 0.5 D maximum of a conventional refractive IOL). The design form has the diffractive structure with symmetric, double blazed phase structures (back to back), consistent surface area ratio, and consistent maximum phase departure within each diffractive zone. Table 4 and FIGS. 13-15 disclose and illustrate the design parameters and performance predictions.

The EDOF IOL is designed with the depth of focus extended beyond 2.5 D. FIG. 13 illustrates the discrete surface phase profile, and Table 4 the specified design parameters for the ring locations and step heights at the ring's trailing edges. The parameters specified in Table 4 particularly apply to material with refractive index of 1.52 at 550 nanometer wavelength. For material with other refractive indices, the step height will need to be adjusted as follows:

h′=h*C,

C=0.184/(n′−1.336),

in which
h′ is the adjusted step height for different refractive index n′,
h is the step height specified in Table 4,
C is an adjusting coefficient,
n is the material refractive index corresponding to Table 4,
n′ is the different material refractive index. The embodied design is enabled for materials having a refractive index from 1.40-1.58.

TABLE 4
Discrete surface structure

| Trifocal IOL, Type A design | | | Trifocal IOL, Type B design | | |
|---|---|---|---|---|---|
| | | Step | | | Step |
| | rj | Height, hj | | rj | Height, hj |
| Zone #, j | (mm) | (um) | Zone #, j | (mm) | (um) |
| 0 | 0.0000 | 0.9715 | 0 | 0.0000 | 0.9715 |
| 1 | 0.5606 | 4.3043 | 1 | 0.5606 | 4.3043 |
| 2 | 0.7928 | 0.9715 | 2 | 0.7928 | 0.9715 |
| 3 | 0.9710 | 4.3043 | 3 | 0.9710 | 4.3043 |
| 4 | 1.1212 | 0.9715 | 4 | 1.1212 | 0.9715 |
| 5 | 1.2536 | 4.3043 | 5 | 1.2536 | 4.3043 |
| 6 | 1.3732 | 0.9715 | 6 | 1.3732 | 0.9715 |
| 7 | 1.4832 | 4.3043 | 7 | 1.4832 | 4.3043 |
| 8 | 1.5856 | 0.9715 | 8 | 1.5856 | 0.8967 |
| 9 | 1.6818 | 4.3043 | 9 | 1.6818 | 3.6421 |
| 10 | 1.7728 | 0.9715 | 10 | 1.7728 | 0.7473 |
| 11 | 1.8593 | 4.3043 | 11 | 1.8593 | 2.9799 |
| 12 | 1.9420 | 0.9715 | 12 | 1.9420 | 0.5978 |
| 13 | 2.0213 | 4.3043 | 13 | 2.0213 | 2.3177 |
| 14 | 2.0976 | 0.9715 | 14 | 2.0976 | 0.4484 |
| 15 | 2.1712 | 4.3043 | 15 | 2.1712 | 1.6555 |
| 16 | 2.2424 | 0.9715 | 16 | 2.2424 | 0.2989 |
| 17 | 2.3115 | 4.3043 | 17 | 2.3115 | 0.9933 |
| 18 | 2.3785 | 0.9715 | 18 | 2.3785 | 0.1495 |
| 19 | 2.4437 | 4.3043 | 19 | 2.4437 | 0.3311 |
| 20 | 2.5071 | 0.9715 | 20 | 2.5071 | 0.0000 |

The performance of Example 3 IOL was evaluated by the embodied modeling and analysis techniques disclosed herein above.

FIGS. 14A, 14B show the simulation of imaging at the model eye retina for the depth of focus of 3.0 D, and for the monofocal IOL design without diffractive phase structures on the surface.

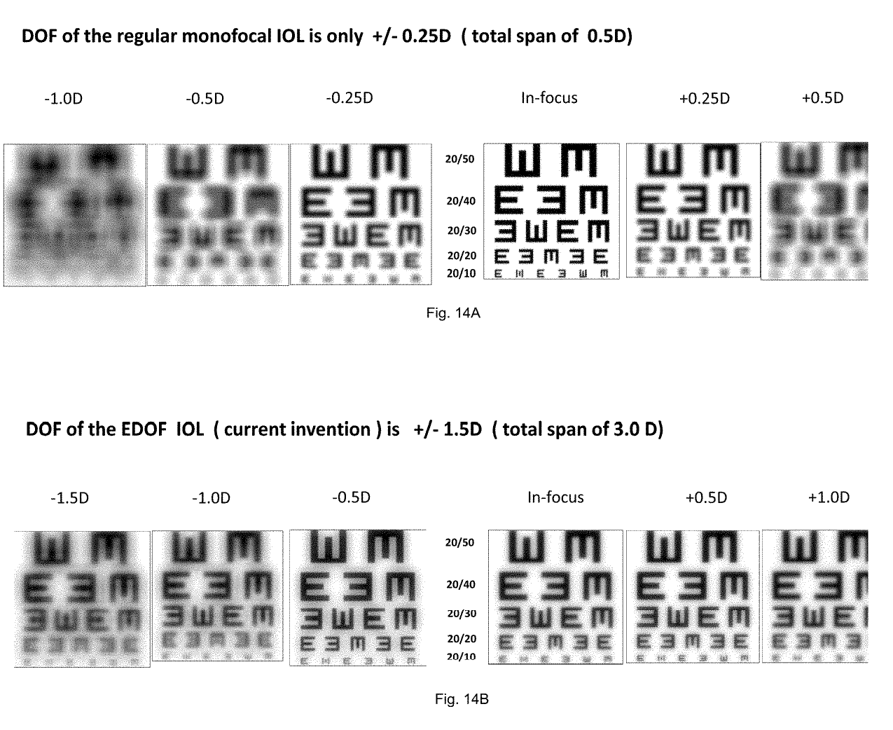

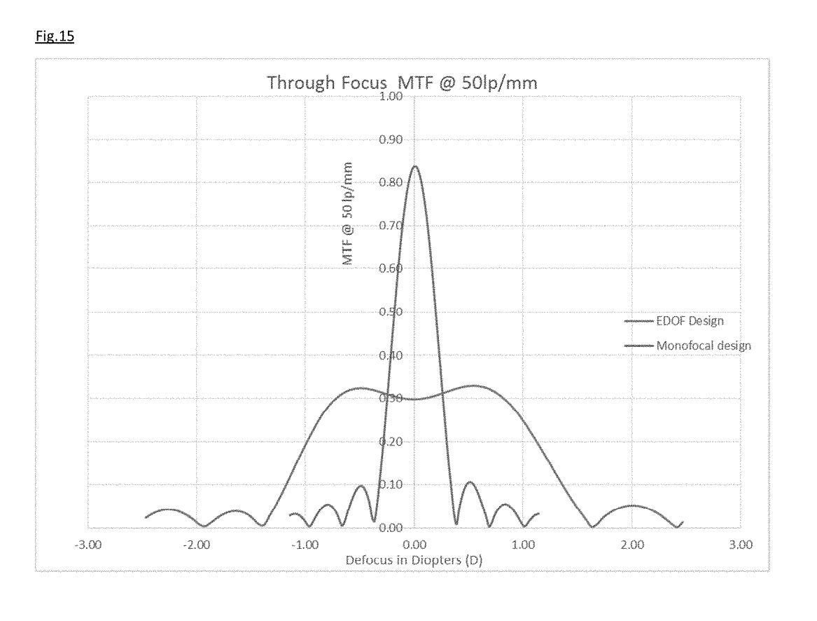

FIG. 15 shows the simulated through-focus MTF curves of the embodied EDOF design and the conventional monofocal IOL.

### Example 4 (Approach IV)

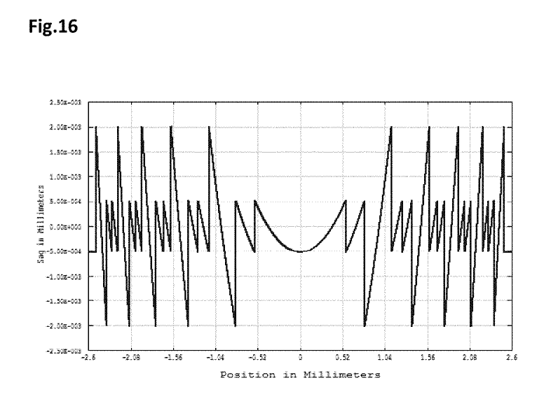

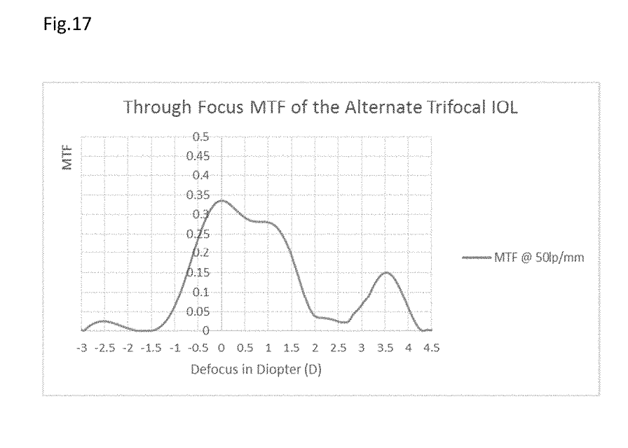

An alternative trifocal design to provide distance, intermediate, and near vision. The design takes the approach of both varied area ratio and varied diffraction efficiencies among the diffractive zones. Different from the trifocal design disclosed in Example 2, this design eliminates the gap from distance to intermediate vision (e.g., about 2.0 D depth of focus at distance vision that creates continuous vision from distance to intermediate vision), and also providing functional near vision. Table 5 and FIGS. 16-17 disclose and illustrate the design parameters and performance predictions.

This alternative trifocal optical design has two distinct add powers, e.g., 1.75 D and 3.50 D to provide distance, intermediate, and near vision. The design takes the approach of both varied area ratio and varied diffraction efficiency among the diffractive zones. The design is targeted to have continuous optical performance from distance to intermediate vision (e.g., about 2.0 D depth of focus at distance vision), and also have functional near vision.

FIG. 16 describes the discrete surface phase structure, and Table 5 the specified design parameters for the ring locations and step heights at the ring's trailing edges. The parameters specified in Table 5 particularly apply to materials having a refractive index of 1.52 at 550 nanometer wavelength. For material with other refractive indices, the step height will need to be adjusted as follows:

h′=h*C,

C=0.184/(n′−1.336),

in which
h′ is the adjusted step height for different refractive index n′,
h is the step height specified in Table 5,
C is an adjusting coefficient,
n is the material refractive index corresponding to Table 5,
n′ is the different material refractive index. The embodied design is enabled for materials of refractive index from 1.40-1.58.

TABLE 5
Discrete surface structure of alternate trifocal design

| | rj | Step Height, hj |
|---|---|---|
| Zone #, j | (mm) | (um) |
| 0 | 0.0000 | 1.0462 |
| 1 | 0.5606 | 1.0462 |
| 2 | 0.7928 | 4.0353 |
| 4 | 1.1212 | 1.0462 |
| 5 | 1.2536 | 1.0462 |
| 6 | 1.3732 | 4.0353 |
| 8 | 1.5856 | 1.0462 |
| 9 | 1.6818 | 1.0462 |
| 10 | 1.7728 | 4.0353 |
| 12 | 1.9420 | 1.0462 |
| 13 | 2.0213 | 1.0462 |
| 14 | 2.0976 | 4.0353 |
| 16 | 2.2424 | 1.0462 |
| 17 | 2.3115 | 1.0462 |
| 18 | 2.3785 | 4.0353 |
| 20 | 2.5071 | 1.0462 |

The performance of Example 4 IOL was evaluated by the embodied modeling and analysis techniques disclosed herein above.

FIG. 17 shows the simulated through-focus MTF curves, and unlike the trifocal design of Example 2, there are no evident MTF fluctuations from distance focus to intermediate focus.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As may be used herein and in the appended claims for purposes of the present disclosure, the term ‘about’ means the amount of the specified quantity plus/minus a fractional amount of or reasonable tolerance thereof that a person skilled in the art would recognize as typical and reasonable for that particular quantity or measurement. Likewise, the term ‘substantially’ means as close to or similar to the specified term being modified as a person skilled in the art would recognize as typical and reasonable as opposed to being intentionally different by design and implementation.

It should also be understood that, unless clearly indicated to the contrary, in any methods claimed herein that include more than one step or act, the order of the steps or acts of the method is not necessarily limited to the order in which the steps or acts of the method are recited.

### APPENDIX 1—THEORETICAL DERIVATION OF EQUATION (1) AND EQUATION (2)

Equation (1) is derived by using one of these two methods, which are detailed in the following:

### Method 1

One method for deriving the radius of the concentric zones is similar to the method used in designing Fresnel Phase Plate, in which the Z locations of the on-axis maximum irradiance is set as the intended focal points, and then the radius of the rings is solved from the on-axis maximum irradiance equation, The mentioned calculation is demonstrated as following:

The on-axis light intensity, I, is determined by the Fresnel number (N_{f}) as

I=2I₀(1+Cos(N_{f}*π)),

in which:
I₀ is constant intensity;
I is the on-axis light intensity corresponding to exact z location;

### N_{f} is the Fresnel Number;

N_{f}=a²/λz=a2/λf.

The on-axis intensity (I) reaches maximum when N_{f}=−2m, . . . −4, −2, 0, 2, 4 . . . 2m (m=integer),

in which:
a is the radius of the Fresnel zone;
A is the wavelength;
z is distance of Z location;
f is focal length.

Make focal length (f) corresponds to the z-location of on-axis maximum irradiance. The radiance of the mth ring can be solved as

aₘ=√2mλf, m=0,1,2 . . . is integer

### Method 2

The second method for deriving the radii of the annular diffractive zones is based on grating equation by Fraunhofer diffraction, however, for diffractive lens, each ring is treated as an individual local grating, and period of local grating is made equal to the diameter of the ring, and the radius of the ring is solved from the grating equation.

Per Fraunhofer diffraction theory, grating equation is expressed as

mλ=Λₘ sin θₘ,

in which:

Λₘ is the grating period of m^{th} diffraction order;

Θₘ is the deflecting angle of m^{th} order diffraction for the rings of the diffraction lens. The radius of the m^{th} ring corresponds to half of the local grating period of the m^{th} annular zone Λₘ, and the grating equation can be expressed as

$$\begin{matrix}m λ = {( {a}_{m}^{2} + {f}^{2} )}^{1 / 2} - f \\ = {a}_{m}^{2} / 2 f .\end{matrix}$$

Then, aₘ²=2m [symbol] f,
therefore aₘ=2mλf, m=0, 1, 2 . . . is integer.

### APPENDIX 2—FUNDAMENTALS OF OPTICAL EVALUATION METHOD FOR DIFFRACTIVE MULTIFOCAL AND EDOF LENSES

Two major theories are adopted to establish the calculation and raytracing method for evaluating the optical performance of diffractive multifocal IOL and EDOF IOL. The established method will generate all the optical performance simulation metrics such MTF, through focus MTF (TF MTF), and imaging simulation

### Theory 1: Coherent Imaging Theory

Coherent imaging is linear with field
The field at image plane Uᵢ(u,v), is the convolution of field at object plane U_{g}(u v) and the amplitude impulse response of the coherent imaging system h(u, v);

Uᵢ(u,v)=h(u,v) [symbol] z,30 U_{g}(u,v).

The amplitude impulse response of the coherent imaging system is the Fourier transform of the pupil function p(x,y);

h(u,v)=FT{p(x,y)}evaluated at frequency fₓ,f_{y}.

Coherent image transfer function (or amplitude transfer function) is the FT of PSF, therefore it is the rescaled pupil function

H(fᵤ,fᵥ)=P(−λZxpfᵤ,−λZxpfᵥ)

### Theory 2: Incoherent Imaging Theory

Incoherent imaging is linear with irradiance. Human eye react with irradiance of the light field Iᵢ(u,v) or I_{g}(u,v).
The irradiance distribution at the image plane is the convolution of PSF (e.g. |h(u,v)|²) and the irradiance distribution of the object

Iᵢ(u,v)=|h(u,v)|² [symbol] [symbol] I_{g}(u,v), therefore

the Optical Transfer Function (OTF) of the incoherent imaging is the Fourier Transform of the PSF, which, according to the derivations using Fourier Transform theory is mathematically equivalent to the auto-correlation of amplitude transfer function, and amplitude transfer function is proportional to the re-scaled pupil function.

H(fᵤ,fᵥ)=H(fᵤ,fᵥ) [symbol] H(fᵤ,fᵥ)=P(−λZxpfᵤ,−λZxpfᵥ) [symbol] P(−λZxpfᵤ,−λZxpfᵥ).

I claim:

1. A multifocal intraocular lens (M-IOL), comprising:
a lens body having an anterior surface and a posterior surface,
wherein at least one of the anterior and the posterior surface is characterized by a discrete phase profile comprising a plurality, m (m=0, 1, 2, 3, . . . ), of contiguous annular, diffractive optical zones each characterized by a radius, rₘ, and a step height, hₘ, at each respective rₘ, wherein at least some values of hₘ may not be equal to hₘ₊ₓ (x=1, 2, 3, . . . ),
wherein rₘ=(2mλf)^{1/2}, where λ is the design wavelength and f is the focal length (1000 mm/Add Power) corresponding to a selected Add Power for the IOL,
further wherein [symbol] ₙ is the diffraction efficiency in a particular optical zone, m, for the n^{th} diffraction order (n=0, 1, 2, 3 . . . ) corresponding to the n^{th} Add Power in that particular optical zone, m, wherein [symbol] ₙ=[sin(π(k−n))/(π(k−n))]²=SINC² (π(k−n))+f(rₘ), where:
k=(n₂−n₁)hₘ/λ is a factor for adjusting the step height, hₘ, where (n₂−n₁) is the refractive index difference between a non-lens medium and the lens optical zone (diffractive) medium, wherein the step height, hₘ, can be determined from the designated [symbol] ₙ,
further wherein an overall energy distribution over a total effective (diffractive) optical area of the IOL is represented as a weighted summation of a local diffraction efficiency [symbol] _{n,m} of the particular optical zone, m (in which n is the diffraction order corresponding to the Add Powerₙ in that m^{th} optical zone,
wherein a weighting factor is determined by a surface area ratio, Rₘ, between the individual optical zone, m, and the total effective (diffractive) optical area of the IOL,
where [symbol] ₙ=(m=1, 2, 3 . . . , n=0, 1, 2, 3 . . . ) and
Rₘ=(area of the m^{th} annular optical zone)/(total effective (diffractive) optical area of IOL).

2. The M-IOL of claim 1, characterized in that [symbol] _{n,m} has a constant value for all of the optical zones, m, and Rₘ has a constant value for all of the optical zones, m.

3. The M-IOL of claim 1, characterized in that [symbol] _{n,m} has a variable value for all of the optical zones, m, and Rₘ has a constant value for all of the optical zones, m.

4. The M-IOL of claim 1, characterized in that [symbol] _{n,m} has a constant value for all of the optical zones, m and Rₘ has a variable value for all of the optical zones, m.

5. The M-IOL of claim 1, characterized in that [symbol] _{n,m} has a variable value for all of the optical zones, m, and Rₘ has a variable value for all of the optical zones, m.

6. The M-IOL of claim 1, characterized in that [symbol] _{n,m} has an adjusting function f(rₘ), which is related to the Fourier Transform of the exact phase profile for the m-th diffraction zone, to optimize light distribution among usable diffraction order and minimize light spreading into unusable diffraction orders.

7. An optical modeling method to simulate the optical performance of a selected M-IOL in an optical ray tracing environment, comprising:
establishing an optical raytracing model eye that can simulate the optical performance of the eye with a selected M-IOL plugged in the model;
constructing a user-defined surface to input a discrete surface phase profile of the selected M-IOL in the optical raytracing model eye, wherein the discrete surface phase profile is associated with a user-defined function that can adjust the phase parameter of each ray traced through the surface based on a local diffractive structure profile.

8. The method of claim 7, further comprising:
tracing rays with phase parameters modified by the diffractive surface to an exit pupil of the raytracing model, and constructing a true pupil function;
determining the Optical Transfer Function (OTF);
determining the modulation transfer function (MTF);
determining the MTF at different defocus locations, which describes the through-focus performance of the design;
determining the system Point Spread Function (PSF); and
conducting imaging simulation.
